(19) United States
(12) Patent Application Publication
(10) Pub. No.: US 2008/0188461 A1
(43) Pub. Date: Aug. 7, 2008

(54) Compositions and methods for detecting, preventing and treating seizures and seizure related disorders

(75) Inventors: Kun-Liang Guan, Ann Arbor, MI (US); David Franz, Springboro, OH (US)

Correspondence Address: Casimir Jones, S.C., 440 Science Drive, Suite 203, Madison, WI, 53711, US

(73) Assignee: Regents of the University of Michigan, Ann Arbor, MI (US)

(21) Appl. No.: 11/823,971

(22) Filed: Jun. 29, 2007

Related U.S. Application Data
(60) Provisional application No. 60/898,856, filed on Feb. 1, 2007.

(51) Int. Cl. A61K 31/55; A61K 31/335; A61P 25/08

(52) U.S. Cl. 514/217; 514/221; 514/450

(57) ABSTRACT

The present invention relates to compositions and methods for the detecting, preventing, treating, and empirically investigating seizures and seizure related disorders (e.g., West syndrome, TSC, childhood absence epilepsy, benign focal epilepsies of childhood, juvenile myoclonic epilepsy (JME), temperol lobe epilepsy, frontal lobe epilepsy, Lennox-Gastaut syndrome, occipital lobe epilepsy). In particular, the present invention provides compositions and methods for detecting, treating, preventing and empirical investigating seizures and seizure related disorders through inhibition of mTOR function. In addition, the present invention provides methods and compositions that utilize mTOR inhibiting agents (e.g., rapamycin) in the detecting, preventing, treating, and empirical investigating of seizures and seizure related disorders.

The present application claims priority to U.S. Provisional Patent Application Ser. No. 60/898,856, filed Feb. 1, 2007, which is herein incorporated by reference in its entirety.

### FIELD OF THE INVENTION

The present invention relates to compositions and methods for the detecting, preventing, treating, and empirically investigating seizures and seizure related disorders (e.g., West syndrome, TSC, childhood absence epilepsy, benign focal epilepsies of childhood, juvenile myoclonic epilepsy (JME), temperol lobe epilepsy, frontal lobe epilepsy, Lennox-Gastaut syndrome, occipital lobe epilepsy). In particular, the present invention provides compositions and methods for detecting, treating, preventing and empirical investigating seizures and seizure related disorders through inhibition of mTOR function (e.g., mTOR activity, mTOR expression). In addition, the present invention provides methods and compositions that utilize mTOR inhibiting agents (e.g., rapamycin) in the detecting, preventing, treating, and empirical investigating of seizures and seizure related disorders.

### BACKGROUND OF THE INVENTION

Seizures, including epileptic seizures, result from a focal or generalized disturbance of cortical function, which may be due to various cerebral or systemic disorders, including, for example, cerebral edema, cerebral hypoxia, cerebral trauma, central nervous system (CNS) infections, congenital or developmental brain defects, expanding brain lesions, hyperpyrexia, metabolic disturbances and the use of convulsive or toxic drugs. It is only when seizures recur at sporadic intervals and over the course of years (or indefinitely) that epilepsy is diagnosed.

Epilepsy is classified etiologically as symptomatic or idiopathic with seizure manifestations that fall into three general categories: 1) generalized tonic-clonic, 2) absence or petiti mal, and 3) complex partial. Symptomatic classification indicates that a probable cause exists and a specific course of therapy to eliminate that cause may be tried, whereas idiopathic indicates that no obvious cause can be found and may be linked to unexplained genetic factors. Of the seizure categories, most persons have only one type of seizure, while about 30% have two or more types.

The risk of developing epilepsy is 1% from birth to age 20 yr. and 3% at age 75 yr. Idiopathic epilepsy generally begins between ages 2 and 14. Seizures before age 2 are usually caused by developmental defects, birth injuries, or a metabolic disease. Those beginning after age 25 may be secondary to cerebral trauma, tumors, or cerebrovascular disease, but 50% are of unknown etiology.

Due to the many interrelationships that exist between the nervous and endocrine systems, defects in synaptic vesicle function can also impact on endocrinological function. For instance, at least two glands secrete their hormones only in response to appropriate neurotransmitter release—the adrenal medulla and the posterior pituitary gland. Upon secretion, hormones are transported in the blood to cause physiologic actions at distant target tissues in the body. Endocrinopathies involving either hyper- or hyposecretion of hormones have pathological consequences. Exemplary of these consequences are giantism and dwarfism, due to hyper- or hyposecretion of growth hormone, respectfully.

A number of techniques are known to treat seizures including, for example, drug therapy, drug infusion into the brain, electrical stimulation of the brain, electrical stimulation of the nervous system, and even lesioning of the brain (see, e.g., U.S. Pat. No. 5,713,923; herein incorporated by reference in its entirety). Current treatments for preventing seizures, however, are successfully in only 60% of cases. As such, improved treatments for preventing seizures are needed.

### SUMMARY OF THE INVENTION

The present invention relates to compositions and methods for the detecting, preventing, treating, and empirically investigating seizures and seizure related disorders (e.g., West syndrome, TSC, childhood absence epilepsy, benign focal epilepsies of childhood, juvenile myoclonic epilepsy (JME), temperol lobe epilepsy, frontal lobe epilepsy, Lennox-Gastaut syndrome, occipital lobe epilepsy). In particular, the present invention provides compositions and methods for detecting, treating, preventing and empirical investigating seizures and seizure related disorders through inhibition of mTOR function (e.g., mTOR activity, mTOR expression). In addition, the present invention provides methods and compositions that utilize mTOR inhibiting agents (e.g., rapamycin, CCI-779, and AP23573) in the detecting, preventing, treating, and empirical investigating of seizures and seizure related disorders.

In experiments conducted during the course of the development of the embodiments of the present invention, inhibition of mTOR function (e.g., through administration of an mTOR inhibiting agent) was shown to reduce the frequency of seizures in individuals suffering from a seizure related disorder. Accordingly, in certain embodiments, the present invention provides methods for treating and/or preventing seizures in a subject, comprising administering to the subject a composition configured to reduce mTOR function (e.g., mTOR activity, mTOR expression) within the subject. In some embodiments, the subject suffers from a seizure related disorder. The composition is not limited to a particular manner of reducing mTOR function (e.g., mTOR activity, mTOR expression) within the subject. In some embodiments, the composition reduces mTOR function through inhibition of at least one of the following components within the subject: PI3K, Akt, LKB1, AMPK, Rheb, mTOR, S6K, 4EBP-1, rS6, e1F4E (e.g., nucleic acid, mRNA, DNA, protein). The composition is not limited to a particular manner of inhibiting such compounds. In some embodiments, the composition comprises an mTOR inhibiting agent (e.g., rapamycin, a rapamycin derivative, or a compound similar in function to rapamycin).

The method is not limited to treating a particular type of seizure related disorder. In some embodiments, the seizure related disorder includes, but is not limited to, West syndrome, TSC, childhood absence epilepsy, benign focal epilepsies of childhood, juvenile myoclonic epilepsy (JME), temperol lobe epilepsy, frontal lobe epilepsy, Lennox-Gastaut syndrome, occipital lobe epilepsy.

In some embodiments, the method further comprises co-administering to the subject an anti-seizure agent. The method is not limited to a particular type or kind of anti-seizure agent, nor is it limited to the administration of a particular number of anti-seizure agents. In some embodiments, the anti-seizure agent is select from at least one of the group consisting of carbamazepine, clobazam, clonazepam, ethosuximide, felbamate, fosphenytoin, flurazepam, gabapentin, lamotrigine, levetiracetam, oxcarbazepine, mephenytoin, phenobarbital, phenytoin, pregabalin, primidone, sodium valproate, tiagabine, topiramate, valproate semisodium, valproic acid, vigabatrin, diazepam, lorazepam, paraldehyde, pentobarbital, and bromides.

In certain embodiments, the present invention provides methods for preventing the onset of seizures in a subject having an increased risk for developing seizures (e.g., an individual suffering from TSC), comprising administering to the subject a composition configured to reduce mTOR function (e.g., mTOR activity, mTOR expression) within the subject. In such embodiments, the composition reduces mTOR function through inhibition of at least one of the following targets within the subject: PI3K, Akt, LKB1, AMPK, Rheb, mTOR, S6K, 4EBP-1, rS6, e1F4E (e.g., nucleic acid, mRNA, DNA, protein). In some embodiments, the composition comprises an mTOR inhibiting agent (e.g., rapamycin, a rapamycin derivative). In some embodiments, the subject suffers from TSC.

The present invention also provides pharmaceutical compositions comprising a pharmaceutically effective amount of an agent that inhibits mTOR function (e.g., mTOR activity, mTOR expression) (e.g., rapamycin, CCI-779, and AP23573), wherein the pharmaceutically effective amount is sufficient to inhibit the frequency of seizures in a subject (e.g., a subject suffering from a seizure related disorder). In some embodiments, the pharmaceutical composition comprises between 1-30 mg of rapamycin (e.g., 1 mg, 2 mg, 3 mg, 5 mg, 10 mg, 15 mg, 20 mg, 29.5 mg rapamycin).

The present invention also provides a kit for characterizing or treating a seizure related disorder in a subject, comprising: a reagent that specifically detects the presence or absence of elevated expression of mTOR; and/or instructions for using the kit for characterizing the disorder in the subject. In some embodiments, the reagent comprises an antibody that specifically binds to mTOR. In some embodiments, the antibody is a monoclonal antibody. In some embodiments, the kit further comprises instructions. In some embodiments, the instructions comprise instructions required by the United States Food and Drug Administration for use in in vitro diagnostic products.

The present invention also provides a method of screening compounds, comprising providing a sample comprising neuron cells having increased mTOR function (e.g., mTOR activity, mTOR expression) (e.g., pyramidal neurons having increased mTOR function, medium spiny neurons of the striatum having increased mTOR function, Purkinje cells having increased mTOR function); and one or more test compounds; and contacting the cell sample with the test compound; and detecting a change in mTOR function in the cell sample in the presence of the test compound relative to the absence of the test compound. In some embodiments, detecting comprises quantifying mTOR mRNA. In other embodiments, detecting comprises quantifying a mTOR polypeptide. In some embodiments, the cell is in vitro. In other embodiments, the cell is in vivo. In some embodiments, the test compound comprises an antisense compound. In other embodiments, the test compound comprises a drug. In some embodiments, the drug is an antibody. In other embodiments, the drug specifically binds to mTOR.

### BRIEF DESCRIPTION OF THE DRAWINGS

FIG. 1 shows a schematic of mammalian target of rapamycin (mTOR) pathway: TSC1 protein, hamartin; TSC2 protein, tuberin; Rheb, Ras homolog enhanced in brain; PTEN, phosphatase and tensin homolog deleted on chromosome 10, 4E-BP1, eukaryotic initiation factor binding protein 1; Raptor, regulatory associated protein of mTor; PKD1, phosphoinositide-dependent protein kinase; IRS, insulin regulated substrate; LST, lethal with sec-thirteen. S6 kinases (S6Ks) are upregulated and 4E-BP1s are downregulated in tuberous sclerosis complex (TSC)-deficient cells as a result of overactivation of mTOR.

### DEFINITIONS

To facilitate an understanding of the present invention, a number of terms and phrases are defined below:

As used herein, the term “seizure” generally refers to temporary abnormal electro-physiologic phenomena of the brain, resulting in abnormal synchronization of electrical neuronal activity. Seizures can manifest as an alteration in mental state, tonic or clonic movements, convulsions, and various other psychic symptoms (such as déjà vu or jamais vu). Seizures are due, for example, to temporary abnormal electrical activity of a group of brain cells.

As used herein, the term “seizure related disorder” refers to any disorder associated with seizures (e.g., an epileptic syndrome disorder). Examples of seizure related disorders include, but are not limited to, West syndrome, TSC, childhood absence epilepsy, benign focal epilepsies of childhood, juvenile myoclonic epilepsy (JME), temperol lobe epilepsy, frontal lobe epilepsy, Lennox-Gastaut syndrome, occipital lobe epilepsy).

As used herein, the term “mTOR pathway,” or “mTOR associated pathway” refers generally to biological (e.g., molecular, genetic, cellular, biochemical, pharmaceutical, environmental) events (e.g., cellular pathways, cellular mechanisms, cellular cascades) involving the mTOR gene and/or the mTOR protein. Examples of components of the mTOR pathway include, but are not limited to, TSC-1, TSC-2, TSC-1/TSC-2, Rheb, mTOR, S6K, and 4EBP-1.

As used herein, the term “mTOR function” refers generally to any type of cellular event for which mTOR is involved (e.g., DNA based activity, mRNA based activity, protein based activity; associated pathway activity) (e.g., mTOR activity, mTOR expression).

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “phosphospecific antibody” refers to an antibody that specifically binds to the phosphorylated form of a polypeptide (e.g., S6K) but does not specifically bind to the non-phosphorylated form of a polypeptide. In some embodiments, phosphospecific antibodies specifically bind to a polypeptide phoshphorylated at a specific position.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5× SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., a seizure related disorder). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids (e.g., blood or urine), solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “effective amount” refers to the amount of a composition (e.g., inhibitor of mTOR) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., compositions of the present invention) to a subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

As used herein, the term “co-administration” refers to the administration of at least two agent(s) (e.g., mTOR siRNAs or antibodies and one or more other agents) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “toxic” refers to any detrimental or harmful effects on a subject, a cell, or a tissue as compared to the same cell or tissue prior to the administration of the toxicant.

As used herein, the term “pharmaceutical composition” refers to the combination of an active agent (e.g., mTOR antibody) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “topically” refers to application of the compositions of the present invention to the surface of the skin and mucosal cells and tissues (e.g., alveolar, buccal, lingual, masticatory, or nasal mucosa, and other tissues and cells that line hollow organs or body cavities).

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975), incorporated herein by reference in its entirety).

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “gene transfer system” refers to any means of delivering a composition comprising a nucleic acid sequence (e.g., mTOR siRNA) to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “transgene” refers to a heterologous gene that is integrated into the genome of an organism (e.g., a non-human animal) and that is transmitted to progeny of the organism during sexual reproduction.

As used herein, the term “transgenic organism” refers to an organism (e.g., a non-human animal) that has a transgene integrated into its genome and that transmits the transgene to its progeny during sexual reproduction.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “restriction endonucleases” and “restriction enzymes” refer to bacterial enzymes, each of which cut double-stranded DNA at or near a specific nucleotide sequence.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene that is used in conjunction with CAD-deficient cells and the mammalian hypoxanthine-guanine phosphoribosyl transferase (hprt) gene that is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp. 16.9-16.15.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “target RNA molecule” refers to an RNA molecule to which at least one strand of the short double-stranded region of an siRNA is homologous or complementary. Typically, when such homology or complementary is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes.

### DETAILED DESCRIPTION OF THE INVENTION

Tuberous sclerosis complex (TSC) is an autosomal dominant genetic disorder with a birth incidence of approximately 1 in 6,000. Affected individuals develop hamartomatous growths in multiple organs of the body that occur throughout their life span. Low-grade neoplastic lesions of the central nervous system (CNS), usually in the form of subependymal giant cell astrocytomas (SEGAs), are reported in 5 to 15% of such individuals. These lesions exhibit insidious slow growth, often remaining clinically asymptomatic until causing obstructive hydrocephalus. This has led to recommendations for periodic neuroimaging of persons with TSC, with resection of SEGAs that exhibit serial growth, cause hydrocephalus, or produce any clinical symptomatology (see, e.g., Torres O A, et al., J Child Neurol 1998; 13: 173-177; Sinson G, et al., Pediatr Neurosurg 1994; 20: 233-239; Cuccia V, et al., Childs Nerv Syst 2003; 19: 232-243; each of which is herein incorporated by reference in their entireties). SEGAs are low-grade astrocytomas (World Health Organization [WHO] grade 1), which do not typically respond to radiation therapy or chemotherapy. Less commonly, more aggressive CNS tumors may occur, in the retina or in other locations (see, e.g., Shields J A, et al., Trans Am Ophthalmol Soc 2004; 102: 139-148; Dashti S R, et al., J Neurosurg 2005; 102(3 suppl): 322-325; Medhkour A, et al., Pediatr Neurosurg 2002; 36: 271-274; each of which are herein incorporated by reference in their entireties). Finally, given the genetic basis of tuberous sclerosis, there is a risk for inducing second malignancies through utilization of standard chemotherapeutic agents or radiation therapy (see, e.g., Matsumura H, et al., Neurol Med Chir (Tokyo) 1998; 38: 287-291; incorporated herein by reference in its entirety).

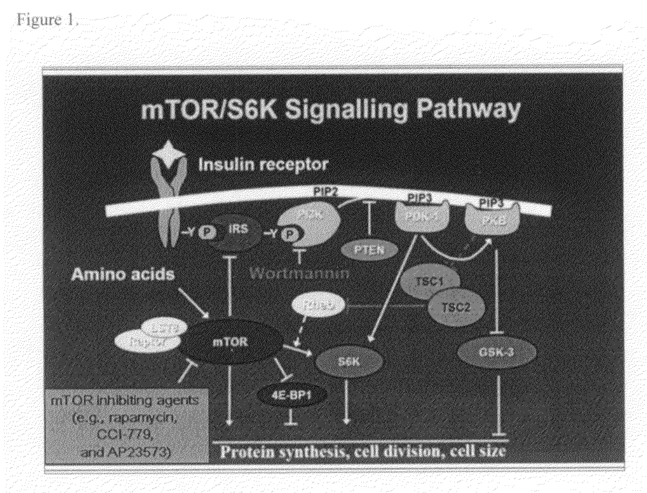

The function of the tuberous sclerosis gene products, hamartin and tuberin, has become increasingly evident over the past several years. Together, they form a tumor suppressor complex, which through the GTPase-activating function of tuberin drives the small GTPase, termed Ras homolog enhanced in brain (Rheb), into the inactive guanosine diphosphate-bound state. Rheb in the guanosine triphosphate-bound active state is a positive effector of the mammalian target of rapamycin (mTOR). mTOR is an evolutionarily conserved protein kinase, which is expressed from fungi to humans. Results over the past 10 years have shown that mTOR serves as a major effector of cell growth as opposed to cell proliferation. Mutations in either hamartin or tuberin drive Rheb into the guanosine triphosphate-bound state, which results in constitutive mTOR signaling. mTOR appears to mediate many of its effects on cell growth through the phosphorylation of the ribosomal protein S6 kinases (S6Ks) and the repressors of protein synthesis initiation factor eIF4E, the 4EBPs. The S6Ks act to increase cell growth and protein synthesis, whereas the 4EBPs serve to inhibit these processes. mTOR interacts with the S6Ks and 4EBPs through an associated protein, Raptor. When mTOR is constitutively activated through mutations in either hamartin or tuberin, this results in the hamartomatous lesions of tuberous sclerosis in the brain, kidney, heart, lung, and other organs of the body (see, e.g., FIG. 1) (see, e.g., Kwiatkowski D J, et al., Cancer Biol Ther 2003; 2: 471-476; Nobukini T, et al., Novartis Found Symp 2004; 262: 148-159, 265-268; each herein incorporated by reference in their entireties). Recent studies have also shown that to function under homeostatic conditions, the mTOR pathway requires both a growth factor/hormone and a nutrient input (see, e.g., FIG. 1). In addition, recent studies have shown that mTOR signaling is also constitutive in neurofibromatosis-associated tumors, and that these effects are also mediated by the de-repression of hamartin/tuberin tumor suppressor complex (see, e.g., Dasgupta B, et al., Cancer Res 2005; 65: 2755-2760; incorporated herein by reference in its entirety). Moreover, it is becoming clear that excessive mTOR signaling is likely to contribute to other forms of nonsyndromic, sporadic human neoplastic diseases, such as breast, prostate, and gastrointestinal cancers (see, e.g., Wu L, et al., Cancer Res 2005; 65: 2825-2831; Rizell M, et al., Anticancer Res 2005; 25(2A): 789-793; Ma L, et al., Cell 2005; 121: 179-193; Asano T, et al., Biochem Biophys Res Commun 2005; 331: 295-302; Roberts L R, et al., Semin Liver Dis 2005; 25: 212-225; Guertin D A, et al., Trends Mol Med 2005; 11: 353-361; each of which are herein incorporated by reference in their entireties). Indeed, lack of expression of hamartin or tuberin was recently suggested to predict poorer outcome and a more aggressive course in human breast cancers (see, e.g., Jiang W G, et al., Eur J Cancer 2005; 41: 1628-1636; Boulay A, et al., Clin Cancer Res 2005; 11: 5319-5328; each of which are herein incorporated by reference in their entireties).

Although other neurologic and systemic manifestations occur, epilepsy is often the most disabling symptom of TSC. Epilepsy in TSC typically involves multiple seizure types, including infantile spasms, and is frequently refractory to available medical and surgical treatments (see, e.g., Curatolo P, et al., Eur J Paediatr Neurol 2002, 6: 15-23; herein incorporated by reference in its entirety).

Cortical tubers, a pathologic hallmark of TSC, often represent the site of seizure onset in TSC patients. The cellular features of tubers, including astrocytosis and abnormally differentiated giant cells with both neuronal and glial features (see, e.g., Crino P B, et al., Neurology 1999;53: 1384-90; incorporated by reference in its entirety), suggest that glial dysfunction may be centrally involved in epileptogenesis in TSC. For example, mice studies have shown that conditional inactivation of the Tsc1 gene in glia results in severe clinical and electroencephalographic seizures by age 2 months and die prematurely by age 4 months (see, e.g., Uhlmann E J, Ann Neurol 2002, 52: 285-96; herein incorporated by reference in its entirety). Pathologically, the brains of these mice exhibit increased astrocyte number and neuronal disorganization within the hippocampus (see, e.g., Uhlmann E J, Ann Neurol 2002, 52: 285-96; herein incorporated by reference in its entirety).

A major function of astrocytes is uptake of extracellular excitatory substances, such as glutamate and potassium (see, e.g., Newman E., Trends Neurosci 2003, 26: 536-42; herein incorporated by reference in its entirety). Elevated levels of extracellular glutamate have been reported in epilepsy patients (see, e.g., During M J, Lancet 1993, 341: 1607-10; Sherwin A, et al., Neurology 1998;38: 920-3; each of which are herein incorporated by reference in their entireties), suggesting that a primary defect in astrocyte glutamate uptake may contribute to seizure formation. In this regard, mice lacking the GLT-1 astrocyte glutamate transporter exhibit frequent seizures (see, e.g., Tanaka K, et al., Science 1997, 276: 1699-702; herein incorporated by reference in its entirety). Moreover, recent studies have demonstrated reduced expression and function of the two primary astrocyte glutamate transporter subtypes, GLT1 and GLAST, in Tsc1^{GFAP}CKO mice (see, e.g., Wong M, et al., Ann Neurol 2003;54: 251-6; herein incorporated by reference in its entirety). In addition to glutamate homeostasis, buffering of extracellular potassium by astrocytes is critical for preventing excessive excitation of neurons (see, e.g., Kojufi P, et al., Neuroscience 2004, 129: 1043-54; herein incorporated by reference in its entirety). Impairment of extracellular potassium uptake by astrocytes via barium-sensitive, inward-rectifier potassium channels (Kir channels) has previously been associated with epilepsy (see, e.g., Bordey A, et al., Epilepsy Res 1998;32: 286-303; Gabriel S, et al., Neurosci Lett 1998;242: 9-12; Gabriel S, et al., Neurosci Lett 1998;249: 91-4; Hinterkeuser S, et al., Eur J Neurosci 2000;12: 2087-96; Jauch R, et al., Brain Res 2002;925: 18-27; Janigro D, et al., J Neurosci 1997;17: 2813-24; Schröder W, Hinterkeuser S, Seifert G, et al. Functional and molecular properties of human astrocytes in acute hippocampal slices obtained from patients with temporal lobe epilepsy. Epilepsia 2000;41(suppl 6):S181-4; each herein incorporated by reference in their entireties).

Dendritic spines are small (sub-micrometer) membranous extrusions that protrude from a dendrite and form one half of a synapse. Typically spines have a bulbous head (the spine head) which is connected to the parent dendrite through a thin spine neck. Dendritic spines are found on the dendrites of most principal neurons in the brain including cortical pyramidal neurons, medium spiny neurons of the striatum and Purkinje cells in the cerebellum. Hippocampal and cortical pyramidal neurons may receive tens of thousands of mostly excitatory inputs from other neurons onto their equally numerous spines, whereas the number of spines on Purkinje neuron dendrites is an order of magnitude larger. Spines come in a variety of shapes and have been categorized accordingly, e.g. mushroom spines, thin spines and stubby spines. Electron microscopy studies have shown that there is a continuum of shapes between these categories. There is some evidence that differently shaped spines reflect different developmental stages and also strengths of a synapse. Using two-photon laser scanning microscopy and confocal microscopy, it has been shown that the volume of spines can change depending on the types of stimuli that are presented to a synapse. Also using the same technique, time-lapse studies in the brains of living animals have shown that spines come and go, with the larger mushroom spines being the most stable over time.

Dendritic spines are believed to restrict diffusion of ions and second messengers from the synapse to the dendrite. As such, they form biochemical compartments that can encode changes in the state of an individual synapse without necessarily affecting the state of other synapses of the same neuron. Changes in dendritic spine density underlie many brain functions, including motivation, learning, and memory. In particular, long-term memory is mediated in part by the growth of new dendritic spines to reinforce a particular neural pathway. By strengthening the connection between two neurons, the ability of the presynaptic cell to activate the postsynaptic cell is enhanced. This type of synaptic regulation forms the basis of synaptic plasticity.

Increased mTOR activity has been shown to alter the morphology of dendritic spines in TSC and non-TSC neurons (see, e.g., Kumar, et al., 2005 J. Neuroscience 25(49):11288-11299; herein incorporated by reference in its entirety). In addition, mTOR has been shown to regulate the synthesis and density of glutamate receptors and other proteins in dendritic spines (see, e.g., Tavazoie, et al., 2005 Nature Neuroscience 8(12):1727; herein incorporated by reference in its entirety).

In experiments conducted during the course of the development of the embodiments of the present invention, inhibition of mTOR function (e.g., through administration of an mTOR inhibiting agent) was shown to reduce the frequency of seizures in individuals suffering from a seizure related disorder. Accordingly, in certain embodiments, the present invention provides methods for treating and/or preventing seizures in a subject, comprising administering to the subject a composition configured to reduce mTOR function (e.g., mTOR activity, mTOR expression) within the subject. In some embodiments, the subject suffers from a seizure related disorder. The composition is not limited to a particular manner of reducing mTOR function within the subject. In some embodiments, the composition reduces mTOR function through inhibition of at least one of the following components within the subject: PI3K, Akt, LKB1, AMPK, Rheb, mTOR, S6K, 4EBP-1, rS6, e1F4E (e.g., nucleic acid, mRNA, DNA, protein). The composition is not limited to a particular manner of inhibiting such compounds. In some embodiments, the composition comprises an mTOR inhibiting agent (e.g., rapamycin, a rapamycin derivative, or a compound similar in function to rapamycin). Exemplary compositions and methods of the present invention are described in more detail in the following sections: I. mTOR Inhibiting Agents; II. Detection of Seizure Related Disorders; III. In vivo Imaging; IV. Antibodies; V. Therapeutics; VI. Pharmaceutical Compositions; VII. Drug Screening; and VIII. Kits.

The practice of the present invention employs, unless otherwise indicated, conventional techniques of organic chemistry, pharmacology, molecular biology (including recombinant techniques), cell biology, biochemistry, and immunology, which are within the skill of the art. Such techniques are explained fully in the literature, such as, “Molecular cloning: a laboratory manual” Second Edition (Sambrook et al., 1989); “Oligonucleotide synthesis” (M. J. Gait, ed., 1984); “Animal cell culture” (R. I. Freshney, ed., 1987); the series “Methods in enzymology” (Academic Press, Inc.); “Handbook of experimental immunology” (D. M. Weir & C. C. Blackwell, eds.); “Gene transfer vectors for mammalian cells” (J. M. Miller & M. P. Calos, eds., 1987); “Current protocols in molecular biology” (F. M. Ausubel et al., eds., 1987, and periodic updates); “PCR: the polymerase chain reaction” (Mullis et al., eds., 1994); and “Current protocols in immunology” (J. E. Coligan et al., eds., 1991), each of which is incorporated herein by reference in their entireties.

I. mTOR Inhibiting Agents

mTOR, is a serine/threonine protein kinase that regulates cell growth, cell proliferation, cell motility, cell survival, protein synthesis, and transcription (see, e.g., Hay N, et al. (2004) Genes & Development, 18(16): 1926-45; Beevers C S, et al. (2006) International Journal of Cancer, 119(4):757-64; each herein incorporated by reference in their entireties). mTOR integrates input from multiple upstream pathways, including insulin, growth factors (such as IGF-1 and IGF-2), and mitogens (see, e.g., Hay N, et al. (2004) Genes & Development, 18(16): 1926-45; herein incorporated by reference in its entirety). mTOR also functions as a sensor of cellular nutrient and energy levels and redox status (see, e.g., Hay N, et al. (2004) Genes & Development, 18(16): 1926-45; Tokunaga C, et al. (2004) Biochemical and Biophysical Research Communications, 313:443-46; Sarbassov D D, et al. (2005) Journal of Biological Chemistry, 280(47):39505-509; each herein incorporated by reference in their entireties). The dysregulation of the mTOR pathway is implicated as a contributing factor to various human disease processes (see, e.g., Beevers C S, et al. (2006) International Journal of Cancer, 119(4):757-64; herein incorporated by reference in its entirety), including but not limited to TSC, epilepsy and diabetes. Rapamycin is a bacterial natural product that can inhibit mTOR through association with its intracellular receptor FKBP12 (see, e.g., Huang S, et al. (2001) Drug Resistance Updates, 4:378-91; Huang S, et al. (2003) Cancer Biology and Therapy, 2:222-232; each herein incorporated by reference in its entirety). The FKBP12-rapamycin complex binds directly to the FKBP12-Rapamycin Binding (FRB) domain of mTOR (see, e.g., Huang S, et al. (2003) Cancer Biology and Therapy, 2:222-232; incorporated herein by reference in its entirety).

mTOR has been shown to function as the catalytic subunit of two distinct molecular complexes in cells (see, e.g., Wullschleger S, et al. (2006) Cell, 124(3):471-84; incorporated herein by reference in its entirety). mTOR Complex 1 (mTORC1) is composed of mTOR, regulatory associated protein of mTOR (Raptor), and mammalian LST8/G-protein β-subunit like protein (mLST8/GβL) (see, e.g., Kim D H, et al. (2002) Cell, 110:163-75; Kim D H, et al. (2003) Molecular Cell, 11:895-904; each incorporated herein by reference in their entireties). This complex possesses the classic features of mTOR by functioning as a nutrient/energy/redox sensor and controlling protein synthesis (see, e.g., Kim D H, et al. (2002) Cell, 110:163-75; Hay N, et al. (2004) Genes & Development, 18(16): 1926-45; each incorporated herein by reference in their entireties). The activity of this complex is stimulated by insulin, growth factors, serum, phosphatidic acid, amino acids (particularly leucine), and oxidative stress (see, e.g., Kim D H, et al. (2002) Cell, 110:163-75; Sarbassov D D, et al. (2005) Journal of Biological Chemistry, 280(47):39505-509; Fang Y, et al. (2001) Science, 294:1942-45; each incorporated herein by reference in their entireties). mTORC1 is inhibited by low nutrient/amino acid levels, serum-starvation/growth factor deprivation, reductive stress, and caffeine, rapamycin, farnesylthiosalicylic acid (FTS) and curcumin (see, e.g., Kim D H, et al. (2002) Cell, 110:163-75; Sarbassov D D, et al. (2005) Journal of Biological Chemistry, 280(47):39505-509; McMahon L P, et al. (2005) Molecular Endocrinology, 19(1):175-83; Beevers C S, et al. (2006) International Journal of Cancer, 119(4):757-64; each incorporated herein by reference in their entireties). Two characterized targets of mTORC1 are p70-S6 Kinase 1 (S6K1) and eukaryotic initiation factor 4E (eIF4E) binding protein 1 (4E-BP1) (see, e.g., Hay N, et al. (2004) Genes & Development, 18(16): 1926-45; incorporated herein by reference in its entirety). mTORC1 phosphorylates S6K1 on at least two residues, with the most critical modification occurring on threonine389 (see, e.g., Saitoh M, et al. (2002) Journal of Biological Chemistry, 277:20104-112; Pullen N, et al. (1997) FEBS Letters, 410:78-82; incorporated herein by reference in its entirety). This event stimulates the subsequent phosphorylation of S6K1 by PDK1 (see, e.g., Pullen N, et al. (1997) FEBS Letters, 410:78-82; Pullen N, et al. (1998) Science, 279:707-10; each incorporated herein by reference in their entireties). Active S6K1 can in turn stimulate the initiation of protein synthesis through activation of S6 Ribosomal protein (a component of the ribosome) and other components of the translational machinery (see, e.g., Peterson R, et al. (1998) Current Biology, 8:R248-50; incorporated herein by reference in its entirety). S6K1 can also participate in a positive feedback loop with mTORC1 by phosphorylating mTOR's negative regulatory domain at threonine2446 and serine2448, events which appear to be stimulatory in regards to mTOR activity (see, e.g., Chiang G G, et al. (2005) Journal of Biological Chemistry, 280:25485-90; Holz M K, et al. (2005) Journal of Biological Chemistry, 280:26089-93; each incorporated herein by reference in their entireties). mTORC1 has been shown to phosphorylate at least four residues of 4E-BP1 in a hierarchial manner (see, e.g., Gingras A C, et al. (1999) Genes & Development, 13:1422-37; Huang S, et al. (2001) Drug Resistance Updates, 4:378-91; Mothe-Satney I, et al. (2000) Journal of Biological Chemistry, 275:33836-43; each incorporated herein by reference in their entireties). Non-phosphorylated 4E-BP1 binds tightly to the translation initiation factor eIF4E, preventing it from binding to 5′-capped mRNAs and recruiting them to the ribosomal initiation complex (see, e.g., Hay N, et al. (2004) Genes & Development, 18(16): 1926-45; Pause A, et al. (1994) Nature, 371:762-67; each incorporated herein by reference in their entireties). Upon phosphorylation by mTORC1, 4E-BP1 releases eIF4E, allowing it to perform its function (see, e.g., Pause A, et al. (1994) Nature, 371:762-67; incorporated herein by reference in its entirety). The activity of mTORC1 appears to be regulated through a dynamic interaction between mTOR and Raptor, one which is mediated by GβL (see, e.g., Kim D H, et al. (2002) Cell, 110:163-75; Kim D H, et al. (2003) Molecular Cell, 11:895-904; each incorporated herein by reference in their entireties). Raptor and mTOR share a strong N-terminal interaction and a weaker C-terminal interaction near mTOR's kinase domain (see, e.g., Kim D H, et al. (2002) Cell, 110:163-75; incorporated herein by reference in its entirety). When stimulatory signals are sensed, such as high nutrient/energy levels, the mTOR-Raptor C-terminal interaction is weakened, allowing mTOR kinase activity to be turned on (see, e.g., Kim D H, et al. (2002) Cell, 110:163-75; incorporated herein by reference in its entirety). When stimulatory signals are withdrawn, such as low nutrient/energy levels, the mTOR-Raptor C-terminal interaction is strengthened, essentially shutting off mTOR kinase function (see, e.g., Kim D H, et al. (2002) Cell, 110:163-75; incorporated herein by reference in its entirety).

mTOR Complex 2 (mTORC2) is composed of mTOR, rapamycin-insenstivie companion of mTOR (Rictor), GβL, and mammalian stress-activated protein kinase interacting protein 1 (mSIN1)(see, e.g., Frias M A, et al. (2006) Current Biology, 16(18):1865-70; Sarbassov D D, et al. (2004) Current Biology, 14:1296-1302; each incorporated herein by reference in their entireties). mTORC2 has been shown to function as an important regulator of the cytoskeleton through its stimulation of F-actin stress fibers, paxillin, RhoA, Rac1, Cdc42, and protein kinase C α (PKCα) (see, e.g., Sarbassov D D, et al. (2004) Current Biology, 14:1296-1302; incorporated herein by reference in its entirety). However, an unexpected function of mTORC2 is its role as “PDK2.” mTORC2 phosphorylates the serine/threonine protein kinase Akt/PKB at serine473, an event which stimulates Akt phosphorylation at threonine308 by PDK1 and leads to full Akt activation (see, e.g., Sarbassov D D, et al. (2004) Current Biology, 14:1296-1302; Stephens L, et al. (1998) Science, 279:710; each incorporated herein by reference in their entireties). mTORC2 appears to be regulated by insulin, growth factors, serum, and nutrient levels (see, e.g., Frias M A, et al. (2006) Current Biology, 16(18):1865-70; incorporated herein by reference in its entirety). Originally, mTORC2 was identified as a rapamycin-insensitive entity, as acute exposure to rapamycin did not affect mTORC2 activity or Akt phosphorylation (see, e.g., Sarbassov D D, et al. (2004) Current Biology, 14:1296-1302; Sarbassov D D, et al. (2005) Science, 307:1098-1101; each incorporated herein by reference in their entireties). However, subsequent studies have shown that chronic exposure to rapamycin, while not effecting pre-existing mTORC2 s, can bind to free mTOR molecules, thus inhibiting the formation of new Complex 2s (see, e.g., Sarbassov D D, et al. (2006) Molecular Cell, 22(2):159-68; incorporated herein by reference in its entirety). It has also been shown that curcumin can inhibit the mTORC2-mediated phosphorylation of Akt/PKB at serine473, with subsequent loss of PDK1-mediated phosphorylation at threonine308 (see, e.g., Beevers C S, et al. (2006) International Journal of Cancer, 119(4):757-64; incorporated herein by reference in its entirety).

The present invention provides agents capable of inhibiting mTOR function (e.g., mTOR activity, mTOR expression). The present invention is not limited to a particular type of agent capable of inhibiting mTOR expresssion. In some embodiments, the mTOR inhibiting agent is an agent that inhibits any part of the pathways associated with mTOR function (e.g., mTOR activity, mTOR expression) (e.g., PI3K, Akt, LKB1, AMPK, TSC-1, TSC-2, TSC-1/TSC-2, Rheb, S6K, 4EBP-1, rS6, e1F4E). In some embodiments, the mTOR inhibiting agent is rapamycin and rapamycin derivatives. In some embodiments, the mTOR inhibiting agent is CCI-779, or AP23573.

Rapamycin (sirolimus [Rapamune]) is a commercially available immunosuppressant, that forms an inhibitory complex with the immunophilin FKBP12, which then binds to and inhibits the ability of mTOR to phosphorylate downstream substrates, such as the S6Ks and 4EBPs. It is marketed as an immunosuppressant, because of its propensity to inhibit T-cell proliferation, and has been approved for use in this therapeutic setting in the United States since 2001. A prodrug for rapamycin, CCI-779 or temsirolimus, is in clinical development for use in a number of therapeutic indications, including oncology (see, e.g., CCI-779, cell cycle inhibitor-779. Drugs RD 2004; 5: 363-367; herein incorporated by reference in its entirety). Animal studies have demonstrated the ability of rapamycin to inhibit the aberrant growth of TSC-deficient cells in vitro and to induce apoptosis of renal tumors in animal models of TSC (see, e.g., Kenerson H, et al., Pediatr Res 2005; 57: 67-75; herein incorporated by reference in its entirety).

In some embodiments, the present invention provides compositions for detecting, treating and empirically investigating seizure related disorders, wherein the compositions comprise mTOR inhibiting agents (e.g., rapamycin and/or rapamycin derivatives). In some embodiments, such compositions comprising mTOR inhibiting agents (e.g., rapamycin and/or rapamycin derivatives) are used to reduce the frequency of seizures in subjects (e.g., subjects suffering from seizure related disorders such as West syndrome, TSC, childhood absence epilepsy, benign focal epilepsies of childhood, juvenile myoclonic epilepsy (JME), temperol lobe epilepsy, frontal lobe epilepsy, Lennox-Gastaut syndrome, occipital lobe epilepsy.

### II. Detection of Seizure Related Disorders

In some embodiments, the present invention provides methods of detecting seizure related disorders (e.g., West syndrome, TSC, childhood absence epilepsy, benign focal epilepsies of childhood, juvenile myoclonic epilepsy (JME), temperol lobe epilepsy, frontal lobe epilepsy, Lennox-Gastaut syndrome, occipital lobe epilepsy) comprising detecting and quantifying mTOR function (e.g., mTOR activity, mTOR expression). In experiments conducted during the course of the development of the embodiments of the present invention, it was shown reduction of mTOR function in individuals suffering from seizure related disorders resulted in a reduction in the frequency of seizures. Accordingly, the embodiments of the present invention provides mTOR as a biomarker for seizure related disorders. The present invention further provides methods of using mTOR biomarkers (e.g., PI3K, Akt, LKB2, AMPK, TSC-1, TSC-2, TSC-1/TSC-2, Rheb, mTOR, S6K, 4EBP-1, rS6, e1F4E) for monitoring, detecting, diagnosing and treating seizure related disorders.

In some embodiments, the present invention provides methods for detecting and quantifying expression of mTOR biomarkers (e.g., PI3K, Akt, LKB1, AMPK, TSC-1, TSC-2, TSC-1/TSC-2, Rheb, mTOR, S6K, 4EBP-1, rS6, e1F4E). In some embodiments, expression is measured directly (e.g., at the nucleic acid level). In some embodiments, expression is detected in tissue samples (e.g., biopsy tumor tissue). In other embodiments, expression is detected in bodily fluids (e.g., including but not limited to, plasma, serum, whole blood, mucus, and urine). The present invention further provides panels and kits for the detection and quantification of mTOR biomarkers (e.g., PI3K, Akt, LKB1, AMPK, TSC-1, TSC-2, TSC-1/TSC-2, Rheb, mTOR, S6K, 4EBP-1, rS6, e1F4E). In preferred embodiments, the increased (or decreased) expression of a mTOR biomarker (e.g., PI3K, Akt, LKB1, AMPK, TSC-1, TSC-2, TSC-1/TSC-2, Rheb, mTOR, S6K, 4EBP-1, rS6, e1F4E) is used to provide a prognosis to a subject (e.g., increased risk for developing seizures).

In some embodiments, detection of the presence or absence of a seizure related disorder or the characterization of a seizure related disorder is accomplished through comparing expression levels of mTOR biomarkers (e.g., PI3K, Akt, LKB1, AMPK, TSC-1, TSC-2, TSC-1/TSC-2, Rheb, mTOR, S6K, 4EBP-1, rS6, e1F4E) over a period of time (e.g., between two time points, three time points, ten time points, etc.). In such embodiments, a change in expression level for a mTOR biomarker (e.g., PI3K, Akt, LKB1, AMPK, TSC-1, TSC-2, TSC-1/TSC-2, Rheb, mTOR, S6K, 4EBP-1, rS6, and/or e1F4E) over a period of time indicates, for example, an increased risk for developing a seizure related disorder, or a change in status for a subject already diagnosed with a seizure related disorder. In such embodiments, a change in expression level for mTOR biomarkers (e.g., PI3K, Akt, LKB1, AMPK, TSC-1, TSC-2, TSC-1/TSC-2, Rheb, mTOR, S6K, 4EBP-1, rS6, e1F4E) over a period of time indicates, for example, a decreased risk for developing a seizure related disorder, or an improved status for a subject already diagnosed with a seizure related disorder (e.g., reduced risk of having additional seizures). In some embodiments, comparing expression of mTOR biomarkers (e.g., PI3K, Akt, LKB1, AMPK, TSC-1, TSC-2, TSC-1/TSC-2, Rheb, mTOR, S6K, 4EBP-1, rS6, e1F4E) over a period of time may be used to test the efficacy of a treatment (e.g., drugs directed toward treating seizure related disorder) and/or may be used to test the efficacy of a new form of treatment (e.g., new drugs directed toward treating a seizure related disorder).

In some embodiments, detection of the presence or absence of a seizure related disorder (e.g., West syndrome, TSC, childhood absence epilepsy, benign focal epilepsies of childhood, juvenile myoclonic epilepsy (JME), temperol lobe epilepsy, frontal lobe epilepsy, Lennox-Gastaut syndrome, occipital lobe epilepsy) or the characterization of a seizure related disorder is accomplished through comparing expression levels of mTOR biomarkers (e.g., PI3K, Akt, LKB1, AMPK, TSC-1, TSC-2, TSC-1/TSC-2, Rheb, mTOR, S6K, 4EBP-1, rS6, e1F4E) to established thresholds. For example, in some embodiments, a subject's expression level for a mTOR biomarker is accomplished through comparing expression levels of mTOR biomarkers (e.g., PI3K, Akt, LKB1, AMPK, TSC-1, TSC-2, TSC-1/TSC-2, Rheb, mTOR, S6K, 4EBP-1, rS6, e1F4E) compared with established mTOR biomarker threshold levels (e.g., established threshold level for low risk for developing seizure related disorder; established threshold level for medium risk for developing seizure related disorder; established threshold level for high risk for developing seizure related disorder; established threshold level for having seizure related disorder versus not having seizure related disorder). Established threshold levels may be generated from any number of sources, including but not limited to, groups of subjects having a seizure related disorder, groups of subjects not having a seizure related disorder, groups of subjects having, etc. Any number of subjects within a group may be used to generate an established threshold (e.g., 5 subjects, 10 subjects, 20, 30, 50, 500, 5000, 10,000, etc.). Threshold levels may be generated with any type or source of bodily sample from a subject (e.g., including but not limited to, plasma, serum, whole blood, mucus, and urine).

The information provided through detection of the mTOR biomarkers (e.g., PI3K, Akt, LKB1, AMPK, TSC-1, TSC-2, TSC-1/TSC-2, Rheb, mTOR, S6K, 4EBP-1, rS6, e1F4E) can also be used to direct a course of treatment. For example, if a subject is found to possess altered expression of a mTOR biomarker (e.g., decreased expression for TSC-1, TSC-2, TSC-1/TSC-2) (e.g., increased expression for PI3K, Akt, LKB1, AMPK, Rheb, mTOR, S6K, 4EBP-1, rS6, and/or e1F4E) treatment may be directed to prevent (e.g., reduce, inhibit) the onset or further occurrence of seizures.

The present invention is not limited to the biomarkers described above. Any suitable marker that correlates with a seizure related disorder or the progression of a seizure related disorder may be utilized in combination with those of the present invention. For example, in some embodiments, biomarkers identified as being up or down-regulated in seizure related disorders (e.g., West syndrome, TSC, childhood absence epilepsy, benign focal epilepsies of childhood, juvenile myoclonic epilepsy (JME), temperol lobe epilepsy, frontal lobe epilepsy, Lennox-Gastaut syndrome, occipital lobe epilepsy) using the methods of the present invention are further characterized using microarray (e.g., nucleic acid or tissue microarray), immunohistochemistry, Northern blot analysis, siRNA or antisense RNA inhibition, mutation analysis, investigation of expression with clinical outcome, as well as other methods disclosed herein. Examples of suitable markers include, but are not limited to, mTOR pathway related compounds (e.g., PI3K, Akt, LKB1, AMPK, TSC-1, TSC-2, TSC-1/TSC-2, Rheb, mTOR, S6K, 4EBP-1, rS6, e1F4E).

In some preferred embodiments, detection of mTOR biomarkers (e.g., including but not limited to, those disclosed herein) is accomplished, for example, by measuring the levels of PI3K, Akt, LKB1, AMPK, TSC-1, TSC-2, TSC-1/TSC-2, Rheb, mTOR, S6K, 4EBP-1, rS6, and/or e1F4E in cells and tissue. For example, in some embodiments, PI3K, Akt, LKB1, AMPK, TSC-1, TSC-2, TSC-1/TSC-2, Rheb, mTOR, S6K, 4EBP-1, rS6, and/or e1F4E can be monitored using antibodies (e.g., antibodies generated according to methods described below). In some embodiments, detection is performed on cells or tissue after the cells or tissues are removed from the subject. In other embodiments, detection is performed by visualizing the mTOR biomarker (e.g., PI3K, Akt, LKB1, AMPK, TSC-1, TSC-2, TSC-1/TSC-2, Rheb, mTOR, S6K, 4EBP-1, rS6, e1F4E) in cells and tissues residing within the subject.

In some embodiments, detection of mTOR biomarkers (e.g., PI3K, Akt, LKB1, AMPK, TSC-1, TSC-2, TSC-1/TSC-2, Rheb, mTOR, S6K, 4EBP-1, rS6, e1F4E) is accomplished by measuring the accumulation of corresponding mRNA in a tissue sample. mRNA expression may be measured by any suitable method, including but not limited to, those disclosed below.

In some embodiments, RNA is detected by Northern blot analysis. Northern blot analysis involves the separation of RNA and hybridization of a complementary labeled probe.

In still further embodiments, RNA (or corresponding cDNA) is detected by hybridization to an oligonucleotide probe). A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In some embodiments, reverse-transcriptase PCR (RT-PCR) is used to detect the expression of RNA (e.g., PI3K, Akt, LKB1, AMPK, TSC-1, TSC-2, TSC-1/TSC-2, Rheb, mTOR, S6K, 4EBP-1, rS6, e1F4E). In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

In some embodiments, detection of mTOR biomarkers (e.g., PI3K, Akt, LKB1, AMPK, TSC-1, TSC-2, TSC-1/TSC-2, Rheb, mTOR, S6K, 4EBP-1, rS6, e1F4E) is accomplished through protein expression. Protein expression may be detected by any suitable method. In some embodiments, proteins are detected by binding of an antibody specific for the protein. The present invention is not limited to a particular antibody. Any antibody (monoclonal or polyclonal) that specifically detects mTOR biomarkers (e.g., PI3K, Akt, LKB1, AMPK, TSC-1, TSC-2, TSC-1/TSC-2, Rheb, mTOR, S6K, 4EBP-1, rS6, e1F4E) may by utilized. In some embodiments, mTOR biomarkers (e.g., PI3K, Akt, LKB1, AMPK, TSC-1, TSC-2, TSC-1/TSC-2, Rheb, mTOR, S6K, 4EBP-1, rS6, e1F4E) are detected by immunohistochemistry. In other embodiments, mTOR biomarkers (e.g., PI3K, Akt, LKB1, AMPK, TSC-1, TSC-2, TSC-1/TSC-2, Rheb, mTOR, S6K, 4EBP-1, rS6, e1F4E) are detected by their binding to an antibody raised against mTOR biomarkers (e.g., PI3K, Akt, LKB1, AMPK, TSC-1, TSC-2, TSC-1/TSC-2, Rheb, mTOR, S6K, 4EBP-1, rS6, e1F4E). In some embodiments, commercial antibodies directed toward mTOR biomarkers (e.g., PI3K, Akt, LKB1, AMPK, TSC-1, TSC-2, TSC-1/TSC-2, Rheb, mTOR, S6K, 4EBP-1, rS6, e1F4E) are utilized. The generation of antibodies is described below.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated.

In other embodiments, the immunoassay is as described in U.S. Pat. Nos. 5,599,677 and 5,672,480; each of which is herein incorporated by reference.

III. In vivo Imaging

In some embodiments, in vivo imaging techniques are used to visualize and quantify the expression of mTOR biomarkers (e.g., PI3K, Akt, LKB1, AMPK, TSC-1, TSC-2, TSC-1/TSC-2, Rheb, mTOR, S6K, 4EBP-1, rS6, e1F4E) in an animal (e.g., a human or non-human mammal). For example, in some embodiments, mTOR biomarker mRNA or protein is labeled using a labeled antibody specific for the biomarker. Specifically bound and labeled antibodies can be quantified and detected in an individual using any in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the biomarkers of the present invention are described below.

The in vivo imaging methods of the present invention are useful in the research use and the diagnosis of seizure related disorder (e.g., West syndrome, TSC, childhood absence epilepsy, benign focal epilepsies of childhood, juvenile myoclonic epilepsy (JME), temperol lobe epilepsy, frontal lobe epilepsy, Lennox-Gastaut syndrome, occipital lobe epilepsy) in cells that contain the biomarkers of the present invention (e.g., neurological cells). In vivo imaging is used to quantify and visualize the presence of a biomarker indicative of a seizure related disorder. Such techniques allow for diagnosis without the use of a biopsy. In some embodiments, the in vivo imaging methods of the present invention are useful for providing prognoses to patients (e.g., patients suffering from epilepsy, TSC). For example, the presence of mTOR biomarkers (e.g., PI3K, Akt, LKB1, AMPK, TSC-1, TSC-2, TSC-1/TSC-2, Rheb, mTOR, S6K, 4EBP-1, rS6, e1F4E) expressed at an amount outside of an established certain threshold may be indicative of a seizure related disorder likely or not likely to respond to certain treatments.

In some embodiments, reagents (e.g., antibodies) specific for the biomarkers of the present invention are fluorescently labeled. The labeled antibodies can be introduced into a subject (e.g., orally or parenterally). Fluorescently labeled antibodies are detected using any suitable method (e.g., using the apparatus described in U.S. Pat. No. 6,198,107, herein incorporated by reference).

### IV. Antibodies

The present invention provides isolated antibodies. In preferred embodiments, the present invention provides monoclonal antibodies that specifically bind to the mTOR biomarkers (e.g., PI3K, Akt, LKB1, AMPK, TSC-1, TSC-2, TSC-1/TSC-2, Rheb, mTOR, S6K, 4EBP-1, rS6, e1F4E). Examples include, but are not limited to, monoclonal antibody against mTOR (e.g., Abcam#s: ab2732, ab2833, ab19207, ab1093, ab34758, ab25880, ab32028), monoclonal antibody against PI3K (e.g., Abcam#s: ab249, ab250, ab40776, ab32089, ab32401), monoclonal and polyclonal antibodies against Akt (e.g., Abcam#s: ab18785, ab28821, ab27773, ab38449, ab39421, ab28422, ab31391, ab35738, ab24831, ab24818), monoclonal antibody against LKB1 (e.g., Abcam#s: ab15095, ab37219), monoclonal and polyclonal antibodies against AMPK (e.g., Abcam#s: ab31958, ab32508, ab32382, ab32112, ab32047, ab3759, ab39644, ab23875, ab3900, ab3760), monoclonal and polyclonal antibodies against TSC1, TSC2, and TSC1/TSC2 (e.g., Abcam#s: ab32936, ab25881, ab25882, ab40872, ab25883), polyclonal antibodies against Rheb (e.g., Abcam#s: ab25873, ab25976), monoclonal and polyclonal antibodies against S6K1 (e.g., Abcam#s: ab19327, ab19279, ab28554, ab24490, ab19380, ab2571, ab24488, ab32529, ab9367, ab36864, ab32525, ab32359, ab9366, ab5231), monoclonal antibody against rS6 (e.g., Abcam# ab10128), monoclonal and polyclonal antibodies against 4EBP1 (e.g., ab37225, ab32130, ab32024, ab25872, ab2606, ab27792), and antibodies against e1F4E. These antibodies, and others, find use in the diagnostic and therapeutic methods described herein.

An antibody against a biomarker of the present invention may be any monoclonal or polyclonal antibody, as long as it can recognize the biomarker. Antibodies can be produced by using a biomarker of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, biomarkers, as such, or together with a suitable carrier or diluent is administered to an animal (e.g., a mammal) under conditions that permit the production of antibodies. For enhancing the antibody production capability, complete or incomplete Freund's adjuvant may be administered. Normally, the biomarker is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 (1975)). As a fusion promoter, for example, polyethylene glycol (PEG) or Sendai virus (HVJ), preferably PEG is used.

Examples of myeloma cells include NS-1,P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a biomarker of the present invention). For example, a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a biomarker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a biomarker of the present invention (further including a gene having a nucleotide sequence partly altered) can be used as the immunogen. Further, fragments of the biomarker protein may be used. Fragments may be obtained by any method including, but not limited to expressing a fragment of the gene, enzymatic processing of the protein, chemical synthesis, and the like.

### V. Therapeutics

In preferred embodiments, the present invention provides a method of preventing, treating and/or researching seizures in subjects (e.g., subject suffering from a seizure related disorder) comprising altering (e.g., reducing, inhibiting) mTOR function (e.g., mTOR activity, mTOR expression). In some embodiments, altering mTOR function comprises providing to a cell a composition comprising a mTOR inhibiting agent (e.g., rapamycin, CCI-779, and AP23573). In some embodiments, altering mTOR function comprises altering (e.g., reducing, inhibiting) agents (e.g., associated pathway agents) that interact with mTOR (e.g., PI3K, Akt, LKB1, AMPK, TSC-1, TSC-2, TSC-1/TSC-2, Rheb, S6K, 4EBP-1, rS6, e1F4E). In some embodiments, altering mTOR function comprises altering (e.g., reducing, inhibiting) genes upregulated or downregulated in response to elevated mTOR function. In some embodiments, altering mTOR function involves a combination of several approaches, including but not limited to, altering mTOR function (e.g., mTOR activity, mTOR expression), altering mTOR associated pathways, and altering transcription of upregulated and/or downregulated in response to elevated mTOR function (e.g., mTOR activity, mTOR expression).

The present invention is not limited by the type of inhibitor used to inhibit mTOR function (e.g., mTOR activity, mTOR expression) for treating a seizure related disorder in a cell. Indeed, any compound, pharmaceutical, small molecule or agent (e.g., antibody, protein or portion thereof) that can alter mTOR function (e.g., mTOR activity, mTOR expression) is contemplated to be useful in the methods of the present invention. In some embodiments, inhibitors used in altering mTOR function (e.g., mTOR activity, mTOR expression) include, but are not limited to, rapamycin.

In some embodiments, altering mTOR function (e.g., mTOR activity, mTOR expression) comprises providing to a cell mTOR specific siRNAs. In some embodiments, altering mTOR function comprises providing to a cell siRNAs specific for components of pathways associated with mTOR function. In some embodiments, altering mTOR function comprises providing to a cell siRNAs specific for mTOR and/or agents associated with mTOR associated pathways (e.g., PI3K, Akt, LKB1, AMPK, TSC-1, TSC-2, TSC-1/TSC-2, Rheb, S6K, 4EBP-1, rS6, e1F4E). The present invention is not limited by the siRNA used. For example, in some embodiments, the present invention provides siRNAs of about 18-25 nucleotides long, 19-23 nucleotides long, or even more preferably 20-22 nucleotides long. The siRNAs may contain from about two to four unpaired nucleotides at the 3′ end of each strand. In preferred embodiments, at least one strand of the duplex or double-stranded region of a siRNA is substantially homologous to or substantially complementary to a target RNA molecule (e.g., (e.g., PI3K, Akt, LKB1, AMPK, TSC-1, TSC-2, TSC-1/TSC-2, Rheb, mTOR, S6K, 4EBP-1, rS6, e1F4E). The present invention is not limited by the target RNA molecule/sequence. Indeed, a variety of target sequences are contemplated to be useful in the present invention including, but not limited to, 18-25 nucleotide stretches of the mTOR mRNA sequence (see, e.g., NCBI Accession No. NM_{—}004958 for mTOR).

In some embodiments, altering mTOR function (e.g., mTOR activity, mTOR expression) comprises providing to the cell an antibody specific for mTOR, or an antibody specific for mTOR associated pathways. In some embodiments, the antibody reduces activity of mTOR in the cell. In some embodiments, altering mTOR function in the cell sensitizes the cell to an additional form of therapeutic treatment (e.g., anticonvulsant therapy). In some embodiments, altering mTOR function inhibits symptoms of a seizure related disorder (e.g., West syndrome, TSC, childhood absence epilepsy, benign focal epilepsies of childhood, juvenile myoclonic epilepsy (JME), temperol lobe epilepsy, frontal lobe epilepsy, Lennox-Gastaut syndrome, occipital lobe epilepsy). In some embodiments, the present invention also provides a method of treating a subject with an epileptic syndrome comprising providing a composition comprising an inhibitor of mTOR; and administering the composition to the subject under conditions such that mTOR function is altered. The present invention is not limited to a particular type or kind of epileptic syndrome (e.g., Infantile spasms (West syndrome), TSC, childhood absence epilepsy, benign focal epilepsies of childhood, juvenile myoclonic epilepsy (JME), temperol lobe epilepsy, frontal lobe epilepsy, Lennox-Gastaut syndrome, occipital lobe epilepsy). In some embodiments, the composition comprising an inhibitor of mTOR is co-administered with an agent configured to treat the epileptic syndrome. The present invention is not limited by type of anti-epilepsy agent co-administered. Indeed, a variety of anti-epilepsy agents are contemplated to be useful in the present invention including, but not limited to, carbamazepine, clobazam, clonazepam, ethosuximide, felbamate, fosphenytoin, flurazepam, gabapentin, lamotrigine, levetiracetam, oxcarbazepine, mephenytoin, phenobarbital, phenytoin, pregabalin, primidone, sodium valproate, tiagabine, topiramate, valproate semisodium, valproic acid, vigabatrin, diazepam, lorazepam, paraldehyde, pentobarbital, and bromides. In some embodiments, the anti-epilepsy agent is a form of surgery (e.g., removal of a benign tumor, removal of hippocampal sclerosis, removal of the front part of either the right or left temperol lobe (e.g., anterior temperoral lobectomy), palliative surgery to reduce the frequency or severity of seizures, and a hemispherectomy). Other examples of anti-epilepsy agents include, but are not limited to, ketogenic diets, vagus nerve stimulation, use of a seizure response dog, and acupuncture.

In some embodiments, the present invention provides methods and compositions suitable for therapy (e.g., drug, prodrug, pharmaceutical, or gene therapy) to alter mTOR gene expression, production, or function (e.g., to inhibit mTOR function).

In some embodiments, the present invention provides compositions comprising expression cassettes comprising a nucleic acid encoding an inhibitor of mTOR (e.g., siRNAs, antibodies, peptides and the like), and vectors comprising such expression cassettes. The methods described below are generally applicable across many species. Any of the vectors and delivery methods disclosed herein can be used for modulation of mTOR function (e.g., mTOR activity, mTOR expression) (e.g., in a therapeutic setting). As disclosed herein, the therapeutic methods of the invention are optimally achieved by targeting the therapy to the affected cells. However, in another embodiment, a mTOR inhibitor can be targeted to cells, e.g., using vectors described herein in combination with well-known targeting techniques, for expression of mTOR modulators.

Furthermore, any of the therapies described herein can be tested and developed in animal models. Thus, the therapeutic aspects of the invention also provide assays for mTOR function.

In some embodiments, viral vectors are used to introduce mTOR inhibitors (e.g., siRNAs, proteins or fragments thereof, etc.) to cells. The art knows well multiple methods of altering the level of expression of a gene or protein in a cell (e.g., ectopic or heterologous expression of a gene). The following are provided as exemplary methods of introducing mTOR inhibitors, and the invention is not limited to any particular method.

In some embodiments, the present invention targets the expression of mTOR and/or pathway related components (e.g., PI3K, Akt, LKB1, AMPK, TSC-1, TSC-2, TSC-1/TSC-2, Rheb, mTOR, S6K, 4EBP-1, rS6, e1F4E) (e.g., for treating seizure related disorder such as TSC, epilepsy). For example, in some embodiments, the present invention employs compositions comprising oligomeric antisense compounds, particularly oligonucleotides, for use in modulating the function of nucleic acid molecules encoding mTOR, ultimately modulating the amount of mTOR expressed. This is accomplished by providing antisense compounds that specifically hybridize with one or more nucleic acids encoding mTOR. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of mTOR. In the context of the present invention, “modulation” means either an increase (stimulation) or a decrease (inhibition) in the expression of a gene.

Introduction of molecules carrying genetic information into cells is achieved by any of various methods including, but not limited to, directed injection of naked DNA constructs, bombardment with gold particles loaded with the constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is incorporated herein by reference in their entireties.

### VI. Pharmaceutical Compositions

The present invention further provides pharmaceutical compositions (e.g., comprising an inhibitor of mTOR function described herein). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

In some embodiments, the invention provides pharmaceutical compositions containing (a) one or more inhibitors of mTOR function (e.g., mTOR activity, mTOR expression) (e.g., antisense compounds, antibodies, etc.) and (b) one or more other anti-seizure agents (e.g., anti-convulsant agents). Examples of such anti-seizure agents are described above. In some embodiments, two or more combined anti-seizure agents (e.g., an inhibitor of mTOR and another anti-seizure agent) may be used together or sequentially.

Dosing may be dependent on severity and responsiveness of the disease state (e.g., stage of the seizure related disorder) to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the treatment (e.g., mTOR siRNA or antibody) is administered in maintenance doses, ranging from 0.01 μg to 100 g per kg of body weight, once or more daily, to once every 20 years.

In experiments conducted during the course of the development of the embodiments of the present invention, rapamycin was shown to reduce the number of seizures for individuals having seizure related disorders. The present invention is not limited to a particular amount of rapamycin for administration to a subject (e.g., 100 mg/day, 90 mg/day, 80 mg/day, 50 mg/day, 25 mg/day, 15 mg/day, 10 mg/day, 5 mg/day, 1 mg/day, 0.1 mg/day, 0.01 mg/day). In some embodiments, the amount of rapamycin for administration is between 1 and 30 mg/day (e.g., 5-15 mg/day) (e.g. 7 mg/day).

### VII. Drug Screening

In some embodiments, the present invention provides drug screening assays (e.g., to screen for new drugs for treating and preventing seizures and seizure related disorders). The screening methods of the present invention utilize mTOR biomarkers (e.g., PI3K, Akt, LKB1, AMPK, TSC-1, TSC-2, TSC-1/TSC-2, Rheb, mTOR, S6K, 4EBP-1, rS6, e1F4E) identified using the methods of the present invention. For example, in some embodiments, the present invention provides methods of screening for compounds that alter (e.g., increase or decrease), directly or indirectly, the presence of mTOR biomarkers (e.g., PI3K, Akt, LKB1, AMPK, TSC-1, TSC-2, TSC-1/TSC-2, Rheb, mTOR, S6K, 4EBP-1, rS6, e1F4E). In some embodiments, candidate compounds are antisense agents (e.g., siRNAs, oligonucleotides, etc.) directed against mTOR or pathways associated with mTOR (e.g., PI3K, Akt, LKB1, AMPK, TSC-1, TSC-2, TSC-1/TSC-2, Rheb, S6K, 4EBP-1, rS6, e1F4E). In other embodiments, candidate compounds are antibodies that specifically bind to a mTOR biomarker (e.g., PI3K, Akt, LKB1, AMPK, TSC-1, TSC-2, TSC-1/TSC-2, Rheb, mTOR, S6K, 4EBP-1, rS6, e1F4E) of the present invention. Also contemplated to be discoverable using the compositions and methods of the present invention are proteins, peptides, peptide mimetics, small molecules and other agents that can be used to treat seizure related disorders.

In one screening method, candidate compounds are evaluated for their ability to alter biomarker presence, activity or expression by contacting a compound with a cell and then assaying for the effect of the candidate compounds on the presence or expression of a mTOR biomarker (e.g., PI3K, Akt, LKB1, AMPK, TSC-1, TSC-2, TSC-1/TSC-2, Rheb, mTOR, S6K, 4EBP-1, rS6, e1F4E). In some embodiments, the effect of candidate compounds on expression or presence of a mTOR biomarker (e.g., PI3K, Akt, LKB1, AMPK, TSC-1, TSC-2, TSC-1/TSC-2, Rheb, mTOR, S6K, 4EBP-1, rS6, e1F4E) is assayed for by detecting the level of biomarker present within the cell. In other embodiments, the effect of candidate compounds on expression or presence of a biomarker is assayed for by detecting the level of mTOR biomarker (e.g., PI3K, Akt, LKB1, AMPK, TSC-1, TSC-2, TSC-1/TSC-2, Rheb, mTOR, S6K, 4EBP-1, rS6, e1F4E) present in the extracellular matrix.

In other embodiments, the effect of candidate compounds on expression or presence of biomarkers is assayed by measuring the level of polypeptide encoded by the biomarkers. The level of polypeptide expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) that bind to proteins that generate biomarkers of the present invention, have an inhibitory (or stimulatory) effect on, for example, biomarker expression and/or biomarker activity, or have a stimulatory or inhibitory effect on, for example, the expression or activity of a biomarker substrate. Compounds thus identified can be used to modulate the activity of target gene products either directly or indirectly in a therapeutic protocol, to elaborate the biological function of the target gene product, or to identify compounds that disrupt normal target gene interactions. Compounds that inhibit or enhance the activity, expression or presence of biomarkers find use in the treatment of seizure related disorder (e.g., West syndrome, TSC, childhood absence epilepsy, benign focal epilepsies of childhood, juvenile myoclonic epilepsy (JME), temperol lobe epilepsy, frontal lobe epilepsy, Lennox-Gastaut syndrome, occipital lobe epilepsy).

In one embodiment, the invention provides assays for screening candidate or test compounds that are substrates of a biomarker. In another embodiment, the invention provides assays for screening candidate or test compounds that bind to or modulate the activity of a biomarker.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 (1994)); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 (1993); Erb et al., Proc. Nad. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33.2059 (1994); Carell et al., Angew. Chem. Int. Ed. Engl. 33:2061 (1994); and Gallop et al., J. Med. Chem. 37:1233 (1994).

Libraries of compounds may be presented in solution (e.g., Houghten, Biotechniques 13:412-421 (1992)), or on beads (Lam, Nature 354:82-84 (1991)), chips (Fodor, Nature 364:555-556 (1993)), bacteria or spores (U.S. Pat. No. 5,223,409; herein incorporated by reference), plasmids (Cull et al., Proc. Nad. Acad. Sci. USA 89:18651869 (1992)) or on phage (Scott and Smith, Science 249:386-390 (1990); Devlin Science 249:404-406 (1990); Cwirla et al., Proc. NatI. Acad. Sci. 87:6378-6382 (1990); Felici, J. Mol. Biol. 222:301 (1991)).

This invention further pertains to novel agents identified by the above-described screening assays. Accordingly, it is within the scope of this invention to further use an agent identified as described herein (e.g., a biomarker modulating agent, an antisense marker nucleic acid molecule, a siRNA molecule, a biomarker specific antibody, or a biomarker-binding substrate) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

### VIII. Kits

In yet other embodiments, the present invention provides kits for the detection, characterization, prevention and/or treatment of seizures and seizure related disorder (e.g., West syndrome, TSC, childhood absence epilepsy, benign focal epilepsies of childhood, juvenile myoclonic epilepsy (JME), temperol lobe epilepsy, frontal lobe epilepsy, Lennox-Gastaut syndrome, occipital lobe epilepsy). In some embodiments, the kits contain antibodies specific for mTOR biomarkers (e.g., PI3K, Akt, LKB1, AMPK, TSC-1, TSC-2, TSC-1/TSC-2, Rheb, mTOR, S6K, 4EBP-1, rS6, e1F4E). In some embodiments, the kits contain mTOR inhibiting agents (e.g., rapamycin, CCI-779, and AP23573). In some embodiments, the kits further contain detection reagents and buffers. In other embodiments, the kits contain reagents specific for the detection of nucleic acids (e.g., DNA, RNA, mRNA or cDNA, oligonucleotide probes or primers). In preferred embodiments, the kits contain all of the components necessary and/or sufficient to perform a detection assay, including all controls, directions for performing assays, and any necessary software for analysis and presentation of results.

### Experimental

The following examples are provided in order to demonstrate and further illustrate certain preferred embodiments and aspects of the present invention and are not to be construed as limiting the scope thereof.

### EXAMPLE I

This example describes experiments conducted during the course of the development of the embodiments of the present invention, showing a reduction in the number of seizures for individuals following treatment with rapamycin. In particular, 14 individuals experiencing multiple daily seizures (4 males with diagnosed TSC, 5 females with diagnosed TSC, 1 male with diagnosed Lennox-Gastaut Syndrome, and 4 females with diagnosed Lennox-Gastaut Syndrome) between the ages of 3-18 were administered rapamycin in combination with an anti-epileptic drug regimen. Of the 14 individuals, all had failed greater than seven anti-epileptic medication treatments. 11 of the 14 individuals had failed vagus nerve stimulation treatments. 4 of the 14 had failed vagus nerve stimulation and epilepsy surgery. Each individual received between up to 7 mg/day of rapamycin. Of individuals diagnosed with TSC, 2 experienced a greater than 90% reduction in seizures, and 5 experienced a greater than 50% reduction in seizures. Of the individuals diagnosed with Lennox-Gastaut Syndrome, 3 experienced a greater than 90% reduction in seizures, and 5 experienced a greater than 50% reduction in seizures. The duration of treatment was between 31 and 32 months.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A method of preventing seizures, comprising administering to a subject suffering from a seizure related disorder a composition comprising an agent, wherein said agent is designed to inhibit mTOR function.

2. The method of claim 1, wherein said seizure related disorder is TSC.

3. The method of claim 1, wherein said seizure related disorder is selected from the group consisting of West syndrome, TSC, childhood absence epilepsy, benign focal epilepsies of childhood, juvenile myoclonic epilepsy (JME), temperol lobe epilepsy, frontal lobe epilepsy, Lennox-Gastaut syndrome, occipital lobe epilepsy.

4. The method of claim 1, wherein said agent is rapamycin.

5. The method of claim 1, wherein said treating results in a reduction of seizures experienced by said subject.

6. The method of claim 1, further comprising administration of a ketogenic diet for said subject.

7. The method of claim 1, further comprising administering at least one anti-epileptic drug to said subject.

8. The method of claim 7, wherein said anti-epileptic drug is selected from the group consisting of carbamazepine, clobazam, clonazepam, ethosuximide, felbamate, fosphenytoin, flurazepam, gabapentin, lamotrigine, levetiracetam, oxcarbazepine, mephenytoin, phenobarbital, phenytoin, pregabalin, primidone, sodium valproate, tiagabine, topiramate, valproate semisodium, valproic acid, vigabatrin, diazepam, lorazepam, paraldehyde, pentobarbital, and bromides.

9. The method of claim 4, wherein the amount of rapamycin administered to said subject is at least 1 mg/day.

10. The method of claim 9, wherein said amount of rapamycin administered to said subject is at least 5 mg/day.

11. The method of claim 10, wherein said amount of rapamycin administered to said subject is 7 mg/day.

12. The method of claim 1, wherein said agent is selected from the group consisting of rapamycin, CCI-779, and AP23573.
